(19) United States
(12) Patent Application Publication
(10) Pub. No.: US 2025/0303131 A1
(43) Pub. Date: Oct. 2, 2025

(54) Therapeutic Medicinal Sleeve and Arc Delivery Device

(71) Applicant: Ronald McCoy, Tallahassee, FL (US)

(72) Inventor: Ronald McCoy, Tallahassee, FL (US)

(21) Appl. No.: 18/624,520

(22) Filed: Apr. 2, 2024

(51) Int. Cl. A61M 37/00

(52) U.S. Cl. CPC A61M 37/00

(57) ABSTRACT

The present invention proposes an arc-shaped device that is configured to deliver medicine through transdermal absorption. The device is designed with a proximal and a distal end. The proximal end is shaped to conform around the ear, while the distal end hosts an absorbent sleeve that is infused with medication. The device is capable of dispensing prescribed or non-prescribed medication to the user's ear, thus allowing for transdermal absorption of the medicine into the user's skin. The device is easy to use, and since it avoids the including of needles, it decreases the potential for infections or other complications caused by traditional injection methods. The invention could prove highly beneficial to individuals who require medication regularly, especially those who may face difficulty in swallowing pills or taking medication orally, as it permits ease of use and added convenience. The proposed arc-shaped device could revolutionize the drug delivery method and provide a more comfortable alternative for users.

### CROSS REFERENCE TO RELATED APPLICATIONS

Application Ser. No. 18/624,520
Filing Date Apr. 4, 2024
Inventor Ronald Jerome McCoy

### SPECIFICATION

The invention is a novel transdermal technology system (TTS) designed to revolutionize drug delivery methods. This innovative system features a dual-component design consisting of an Arc Pre-Delivery Device (APDD) and a Therapeutic Dispensing Unit (TDU) for precise and efficient administration of pharmaceutical agents. By utilizing a unique placement around the back of the human ear, the TTS offers targeted drug delivery and cost savings through reduced dosage requirements. With a focus on enhancing patient experience and convenience, this invention has the potential to significantly impact the field of transdermal drug delivery.

### FEDERAL RESEARCH OR DEVELOPMENT

N/A

### REFERENCE TO SEQUENCE LISTING

N/A

### BACKGROUND

This invention relates to an innovative transdermal technology system (TTS) device to treat various medical conditions, such as migraines, high blood pressure, nausea, smoking cessation, and numerous other conditions.

This TTS, known as the Arc Pre-Delivery Device (APDD) and Therapeutic Dispensing Unit (TDU), represents a significant advancement in transdermal drug delivery technology. Comprising two distinct components, the APDD and TDU, this system is designed to provide a comfortable and secure method of administering pharmaceutical agents behind the user's ear.

The APDD, serving as the upper component of the device, features a distinctive arc-like shape with a flexible tip at its proximal end to ensure optimal and secure placement over the top of the human ear. Within the APDD lies a hollow cavity with a centrally positioned slot specifically designed to accommodate a tube containing the pharmaceutical agent. The distal end of the APDD includes an aperture for seamless connection to the TDU, facilitating the effective transdermal drug delivery process.

Complementing the APDD, the TDU functions as the lower component of the system. Its proximal end is strategically engineered to link with the distal end of the APDD, forming a cohesive unit. The interior of the TDU's proximal end houses a puncturing mechanism that, upon connection to the APDD, punctures the tube within the APDD, allowing the pharmaceutical contents to flow into a reservoir within the TDU.

Noteworthy is the absorbent application mechanism located at the tip of the TDU's distal end, serving as the point of contact with the user's skin. This application mechanism becomes saturated with the fluid from the APDD, enabling efficient transdermal administration of the pharmaceutical agent into the skin behind the user's ear lobe.

The design of the TTS system, as described above, not only ensures effective drug delivery but also enables the APDD and TDU components to be reused at daily or medically prescribed intervals, setting a new standard for convenience and sustainability in transdermal drug administration.

Patients who opt for TTS for the delivery of their pharmaceutical agents can expect to achieve cost savings compared to taking these medications in pill form. As TTS bypasses the gastrointestinal system, it necessitates smaller dosages to produce equivalent therapeutic effects, leading to financial benefits for both the patients and the pharmaceutical company.

### History of Transdermal Patches

Transdermal patches have a history that dates back to 1979 when the first nicotine patch was patented. This breakthrough in drug delivery was a result of the innovative research by Dr. Alejandro Zaffaroni, who pioneered the concept of using a patch to deliver medication through the skin. These early patches were designed to help smokers quit by providing a steady release of nicotine into the bloodstream, thereby reducing withdrawal symptoms. This innovation paved the way for the development of various transdermal patches for delivering different medications, including hormone replacement therapy, pain relief, and motion sickness.

The 1980s and 1990s saw significant advancements in transdermal patch technology. with pharmaceutical companies investing in research and development to introduce new patches for different medical conditions. The development of effective adhesive systems and the use of different materials for patch construction further improved the reliability and effectiveness of transdermal drug delivery. This period also marked the introduction of the first FDA-approved transdermal patch for estrogen replacement therapy, addressing the needs of menopausal women and demonstrating the potential of this delivery system for hormone therapy.

In recent years, transdermal patches have continued to evolve, with innovations in patch design, drug formulation, and delivery systems. The growing interest in transdermal drug delivery has led to the development of patches for an even wider range of medications, including cardiovascular drugs, pain management, and neurologic disorders. The history of transdermal patches reflects a remarkable journey of innovation, from the early days of nicotine patches to the diverse array of patches available today, offering patients an alternative and convenient method of drug administration.

### SUMMARY

The transdermal technology system (TTS) described in the present disclosure introduces an innovative approach to drug delivery through a unique dual-component design. The system comprises an Arc Pre-Delivery Device (APDD) and a Therapeutic Dispensing Unit (TDU), offering a more targeted and efficient method of administering pharmaceutical agents. The APDD features an arc-like shape designed for secure placement around the back of the human ear, housing a hollow cavity with a slot to accommodate a pharmaceutical agent-containing tube. The TDU connects to the APDD and includes a puncturing mechanism to allow the flow of drug content from the APDD to the TDU for transdermal application. This design not only enhances drug delivery precision but also introduces a reusable and cost-effective aspect to transdermal drug administration.

In contrast to pill administration of medication, TTS bypasses the gastrointestinal system and thus requires smaller dosages to achieve similar therapeutic effects. The smaller dosage results in a cost savings for both consumers and pharmaceutical companies.

The efficient transdermal delivery method of the present disclosed TTS offers a more convenient and comfortable experience for patients compared to traditional patch applications which are often bulky and conspicuously located.

A review of the detailed description in conjunction with the accompanying drawings adds further clarity and insight into the innovative design and functionality of this cutting-edge and sustainable TTS solution, solidifying its potential to revolutionize the field of transdermal drug delivery.

### DESCRIPTION OF VIEWS

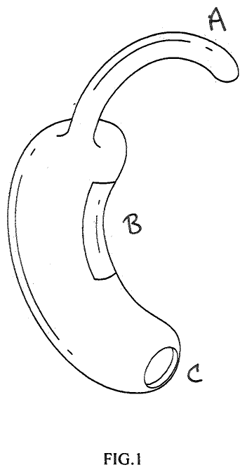

FIG. 1 depicts the Arc Pre-Delivery Device (APDD) in closed position. The element highlighted as (A) is a flexible component which will allow the consumer to comfortably and securely adjust the Device over their ear. The element highlighted as (B) is a mechanism to the APDD to expose the interior of the Device. The element highlighted as (C) is an aperature to facilitate the connection of the APDD to the Therapeutic Dispensing Unit (TDU).

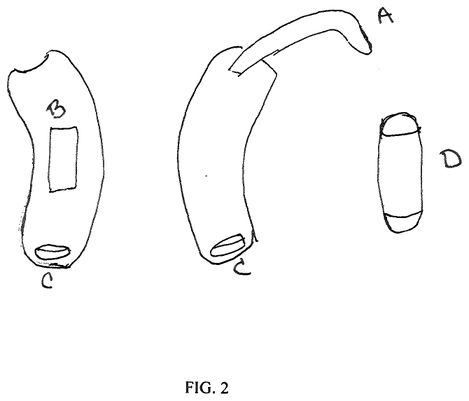

FIG. 2 depicts the APDD in open position. The element highlighted as (A) on the right-side of the APDD is the flexible component as described in FIG. 1A. The element highlighted as (B) on the left-side of the APDD is a slot to house a tube containing a pharmaceutical agent as described in FIG. 2D. The element highlighted as (C) is the aperature as described in FIG. 1C. The element highlighted as D depicts a tube containing a pharmaceutical agent, The bottom of the tube is fitted with a material that capable of being punctured by the TDU upon connection but is secure enough to hold fluid in place prior to connection. The top of the tube is also fitted with a secure material to ensure pharmaceutical agent does not leak or spill prior to use by the consumer.

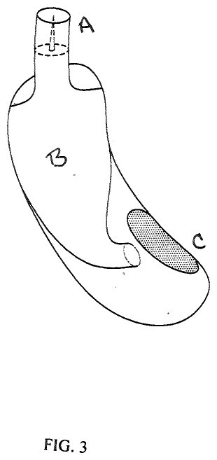

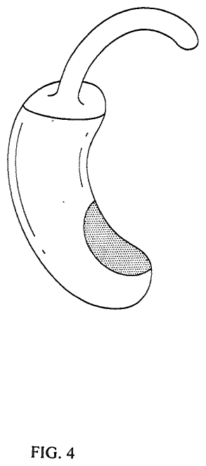

FIG. 3 depicts the Therapeutic Dispensing Unit (TDU). The element highlighted as (A) is the puncturing mechanism which upon connection to the APDD via the aperature at the bottom of the APDD, the pointed device punctures the lower portion of the tube containing the pharmaceutical agent, thereby releases the contents down into the TDU. The element highlighted as (B) is a chamber that receives the pharmaceutical agent released by the APDD. At the distal end of the chamber is an aperature that facilitates passage of the pharmaceutical agent to the application mechanism as described in 3C. The element highlighted as C is an application mechanism that has an absorbent feature which enables the pharmaceutical agent to saturate the mechanism. Upon placement of the TTS System (FIG. 4) on the ear of the consumer, the application mechanism is positioned behind the ear of the consumer touching the skin, allowing for transdermal administration of the pharmaceutical agent.

FIG. 4 depicts both the APDD and the TDU connected. When both are connected, it is a Transdermal Technology System (TTS).

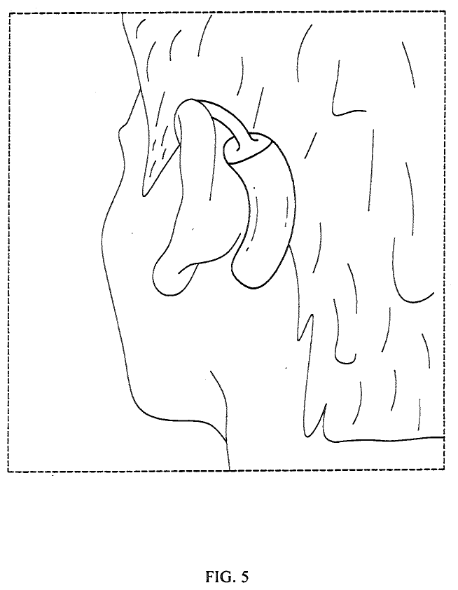

FIG. 5 depicts how the TTS would be worn by the consumer.

### DETAILED DESCRIPTION

The TTS system disclosed is named Arc Pre-Delivery Device (APDD) and Therapeutic Dispensing Unit (TDU). The device is comprised of two components.

The APDD is the upper or top component of the device with a proximal and distal end. The APDD has an arc-like shape with a flexible tip on proximal end (see FIG. 1). The proximal tip has a smaller circumference of roughly eight to ten millimeters than the remainder of the APDD. It is oriented in this manner to ensure a secure placement and comfortable user experience over the human ear. The interior of the APDD features a hollow cavity with a centrally positioned slot designed to accommodate a tube containing a pharmaceutical agent (see FIG. 2) with a proximal and distal end covered by a thin membrane. Residing on the side of the APDD is an access mechanism designed to facilitate opening and closing the device with ease for the user to access the interior cavity. The distal end of the APDD features an aperture to facilitate the device's connection to the TDU (see FIG. 1).

The TDU is the lower of bottom component of the device with a proximal and distal end. The proximal end of the TDU is designed to connect to the distal end of the APDD (see FIG. 3). The interior of the proximal end houses a puncturing mechanism (see FIG. 4). To ensure user safety, the puncture mechanism is thin metal rod housed within a cylindrical chamber that leads to a reservoir. Upon connection to the APDD, the puncture mechanism ruptures the thin membrane on the distal end of the tube causing the contents to flow through the chamber and down into the reservoir within the TDU.

Residing on the tip of the distal end of the TDU is an absorbent application mechanism on the skin-contact side of the device (see FIG. 5). Upon the fluid draining into the reservoir, a small orifice in the reservoir facilitates the passage of the fluid to application mechanism and saturates it.

Upon placement of the TTS (comprising both the APDD and TDU components) around the ear, the TTS begins transdermal administration of the pharmaceutical agent into the skin behind the user's ear lobe (see FIG. 6).

At the conclusion of transdermal administration, both the tube inside the cavity of the APDD and the application mechanism on the TDU should be disposed. To facilitate the attaching and detaching of the application mechanism, TDU will be designed in such a way for it to be easily applied and removed from the device in a secure and safe manner.

The TTS is designed in the above-described manner to enable the APDD and the TDU to be reused on a daily or other medically prescribed interval.

### Differentiation From Transdermal Patches

The APDD and TDU TTS differs from traditional transdermal patches in its innovative design and application method.

First, transdermal patches are often placed on areas of the body where the dermal layer is thick-upper back, forearm, thighs, etc. Contrast that with the APDD and the TDU administrates the pharmaceutical agent behind the ear where the skin is very thin, which enables the medication to reach the bloodstream faster allowing quicker efficacy.

Secondly, patches can often be bulky and conspicuously located. The APDD and TDU TTS is discreetly located behind the ear of the consumer offering greater privacy and convenience.

1: A transdermal therapeutic system (TTS) comprised of two interlocking components.
a. An Arc Pre-Delivery Device (APDD) serving as the upper component, said APDD featuring an arc-like shape with a proximal and distal end, the proximal end of the APDD having an approximate 45-degree angle and having approximate diameter of eight to ten millimeters to secure placement over the top of the human ear, the APDD containing a hollow cavity with a centrally positioned slot designed to accommodate a pharmaceutical agent-containing tube with the proximal and distal end of the tube covered by a thin membrane, and the distal end of the APDD featuring an aperture for connection to the Therapeutic Dispensing Unit (TDU) or lower component.
b. A Therapeutic Dispensing Unit (TDU) serving as the lower component, said TDU designed to connect to the distal end of the APDD, comprising a puncturing mechanism housed within its proximal end upon which when connected to APDD the distal end of the tube's membrane is punctured and subsequently releases the pharmaceutical contents to flow into the TDU, to be absorbed by the application mechanism on the skin-contact side of the TDU's distal end for efficient transdermal administration of the pharmaceutical agent behind the user's ear lobe.

2: The transdermal therapeutic system of claim 1, wherein the APDD and the TDU are configured for reusability at daily or medically prescribed intervals, thereby promoting sustainability and cost-effectiveness in transdermal drug delivery.
